(19) United States
(12) Patent Application Publication
(10) Pub. No.: US 2026/0010982 A1
(43) Pub. Date: Jan. 8, 2026

(54) SYSTEMS AND METHODS FOR ENHANCING RETINAL COLOR FUNDUS IMAGES FOR RETINOPATHY ANALYSIS

(71) Applicants: ARIZONA BOARD OF REGENTS ON BEHALF OF ARIZONA STATE UNIVERSITY, Scottsdale, AZ (US); MAYO FOUNDATION FOR MEDICAL EDUCATION AND RESEARCH, Rochester, MN (US)

(72) Inventors: Wenhui Zhu, Tempe, AZ (US); Peijie Qiu, University City, MO (US); Oana Dumitrascu, Scottsdale, AZ (US); Yalin Wang, Tempe, AZ (US)

(73) Assignees: ARIZONA BOARD OF REGENTS ON BEHALF OF ARIZONA STATE UNIVERSITY, Scottsdale, AZ (US); MAYO FOUNDATION FOR MEDICAL EDUCATION AND RESEARCH, Rochester, MN (US)

(21) Appl. No.: 19/329,945

(22) Filed: Sep. 16, 2025

Related U.S. Application Data
(63) Continuation of application No. PCT/US2024/021838, filed on Mar. 28, 2024.
(60) Provisional application No. 63/492,852, filed on Mar. 29, 2023.

(51) Int. Cl. G06T 5/60; G16H 50/20

(52) U.S. Cl. CPC G06T 5/60; G16H 50/20; G06T 2207/30041

(57) ABSTRACT

An image enhancement method includes translating a first image to a second image by applying a machine learning framework to map a source domain to a target domain. The machine learning network can include two stages: (1) optimal transport guided unpaired image-to-image translation and (2) regularization by enhancing. The first stage can utilize generative adversarial networks (GANs) to map the source domain to the target domain.

### CROSS-REFERENCE TO RELATED APPLICATION

This application is a continuation of PCT Application No. PCT/US2024/21838 filed Mar. 28, 2024, now WIPO Publication No. WO/2024/206542 entitled “SYSTEMS AND METHODS FOR ENHANCING RETINAL COLOR FUNDUS IMAGES FOR RETINOPATHY ANALYSIS.” PCT Application No. PCT/US2024/21838 claims priority to, and the benefit of, U.S. Provisional Patent Application No. 63/492,852, filed on Mar. 29, 2023, entitled “SYSTEMS AND METHODS FOR ENHANCING RETINAL COLOR FUNDUS IMAGES FOR RETINOPATHY ANALYSIS.” The disclosure of each of the foregoing is incorporated herein by reference in its entirety, including but not limited to those portions that specifically appear hereinafter, but except for any subject matter disclaimers or disavowals, and except to the extent that the incorporated material is inconsistent with the express disclosure herein, in which case the language in this disclosure shall control.

### TECHNICAL FIELD

The present disclosure relates to imaging, and in particular to techniques for enhancing images of the retina.

### BACKGROUND

Retinal fundus photography is used to diagnose various ocular diseases. Real-world non-mydriatic retinal fundus photography is prone to artifacts, imperfections, and low-quality when certain ocular or systemic co-morbidities exist. Artifacts may result in inaccuracy or ambiguity in clinical diagnoses. Accordingly, improved approaches remain desirable.

### SUMMARY

In an aspect, an image enhancement method is disclosed. The image enhancement method includes translating a first image to a second image by applying a machine learning framework to map a source domain to a target domain.

In another aspect, a computerized image processing method is disclosed. The computerized image processing method includes applying, by a processor, a machine learning framework to translate a first image to a second image, wherein the first image comprises a source domain and the second image comprises a target domain. The computerized image processing method can further includes saving, by the processor, the second image to a memory.

These and other embodiments may optionally include one or more of the following features.

The machine learning framework can be a generative adversarial network. The machine learning framework can be an optimal transport-guided unpaired generative adversarial network.

The first image and/or the second image can be a retinal fundus photography image.

The method can further include utilizing the second image to assist in diagnosis of retinopathy.

The method can further include classifying the second image into at least one of a plurality of classifications, the plurality of classifications includes a normal classification and one or more disorder classifications, wherein the one or more disorder classifications includes at least one of: age-related macular degeneration (AMD), Diabetic Retinopathy (DR), glaucoma, or Retinal Vein Occlusion (RVO).

The machine learning framework can utilize the equation:

$${max}_{{G}_{θ}} {min}_{{D}_{w}} {\sum }_{i = 1}^{n} [ {αℒ}_{d} ( {y}_{i} , {G}_{θ} ( {y}_{i} ) ) + {βℒ}_{idt} ( {x}_{i} , {G}_{θ} ( {x}_{i} ) ) ] + {𝒲}_{1} ( {ℙ}_{X} , {ℙ}_{{G}_{θ} ( Y )} )$$

where G_{θ} is a generator parameterized by θ, D_{w}, a discriminator, is a 1-Lipschitz function parameterized by w, L_{d} and L_{idt} denotes a domain transport cost and an identity constraint cost, respectively, and a, B are weight parameters of a domain loss and an identity loss, respectively.

The translating can include use of an algorithm of the form:

Algorithm 1 OT-Guided Unpaired Image-to-Image Translation.

```
Require: The learning rate η, the batch size m, the gradient penalty weight
  λ, the consistency loss weight α ≤ 1, the identity loss weight β.
Require: Initial discriminator parameters w₀, initial generator parameters θ₀.
  while not converge do
    $Sample a batch of low ‐ quality images y = {\{ {y}_{i} \}}_{i = 1}^{m} ∼ {ℙ}_{Y} with {\{ {g}_{i} \}}_{i = 1}^{m} .$
    $Sample a batch of high ‐ quality images x = {\{ {x}_{i} \}}_{i = 1}^{m} ∼ {ℙ}_{X} with {\{ {g}_{i} \}}_{i = 1}^{m} .$
    for i = 1, . . . , m do
      Sample a random ϵ~U[0, 1].
      \tilde{x}ᵢ ← G_{θ}(yᵢ)
      \hat{x}ᵢ ← ϵxᵢ + (1 − ϵ) \tilde{x}ᵢ
      ${ℒ}_{{D}_{w}} ( i ) ← {D}_{w} ( {\tilde{x}}_{i} ) - {D}_{w} ( {x}_{i} ) + {λ ( {\vert \vert {∇}_{{\hat{x}}_{i}} {D}_{w} ( {\hat{x}}_{i} ) \vert \vert}_{2} - 1 )}_{+}^{2}$
    end for
    $w ← w + η \cdot RMSProp ( w , {∇}_{w} \frac{1}{m} {\sum }_{i = 1}^{m} {ℒ}_{{D}_{w}} ( i ) )$
    ${ℒ}_{{G}_{θ}} ← \frac{1}{m} {\sum }_{i = 1}^{m} - {D}_{w} ( {G}_{θ} ( y ) ) + α {ℒ}_{d} ( y , {G}_{θ} ( y ) ) + β {ℒ}_{idt} ( x , {G}_{θ} ( x ) )$
    θ ← θ − η · RMSProp(θ, ∇_{θ} [symbol] G_{θ})
  end while
```

The machine learning framework can utilize the equation:

$$\hat{x} = {arg min}_{x} {𝔼}_{x} [ ℒ ( x , y ) ] + γ R ( x ) with R ( x ) = \frac{1}{2} {x}^{T} ( x - {G}_{θ} ( x ) )$$

where γ controls a regularization strength, and L denotes a multi-scale structural similarity loss.

The translating can include use of an algorithm of the form:

Algorithm 2 Regularization by Enhancing.

```
Require: The step size η, regularization strength γ, tolerance tol, Generator
  G_{θ}
Require: Initial \tilde{x}⁽⁰⁾, s⁽⁰⁾ = \tilde{x}⁽⁰⁾, t⁽⁰⁾ = 1
  while not converge do
    ${t}^{( k )} = \frac{1}{2} ( 1 + \sqrt{1 + 4 {( {t}^{( k - 1 )} )}^{2} )}$
    $Der ( {s}^{( k - 1 )} ) = {∇}_{{s}^{( k - 1 )}} ℒ ( {s}^{( k - 1 )} , y ) + γ ( {s}^{( k - 1 )} - {G}_{θ} ( {s}^{( k - 1 )} ) )$
    \tilde{x}^{(k)} ← s^{(k-1)} − η · Der(s^{(k-1)})
    ${s}^{( k )} ← {\tilde{x}}^{( k )} + \frac{{t}^{( k - 1 ) - 1}}{{t}^{( k )}} ( {\tilde{x}}^{( k )} - {\tilde{x}}^{( k - 1 )} )$
    if ||\tilde{x}^{(k)} − \tilde{x}^{(k-1)}|| ≤ tol · ||\tilde{x}^{(k-1)}|| then
      break
    end if
  end while
```

The image enhancing method can further include providing a report indicative of at least one classification of the second image.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described.

### BRIEF DESCRIPTION OF THE DRAWINGS

With reference to the following description and accompanying drawings:

FIG. 1 illustrates a network architecture of an exemplary method for image processing, in accordance with an exemplary embodiment;

FIG. 2 illustrates enhanced images during training at different epochs, in accordance with an exemplary embodiment;

FIG. 3 illustrates the results of an exemplary method as compared to certain prior approaches;

FIG. 4 illustrates a framework of an exemplary method for image processing, in accordance with an exemplary embodiment;

FIG. 5 illustrates the results of an exemplary method as compared to certain prior approaches;

FIG. 6A illustrates the results of an exemplary method including deg2high model evaluation based on the full-reference experiment as compared to certain prior approaches;

FIG. 6B illustrates the results including downstream segmentation tasks of an exemplary method as compared to certain prior approaches;

FIG. 7 illustrates the results of an exemplary method including enhanced images of a low2high model as compared to certain prior approaches;

FIG. 8 schematically illustrates a computer control system or platform programmed or otherwise configured to implement the methods provided herein, in accordance with an exemplary embodiment;

FIG. 9 is a flow chart for a method for image enhancement, in accordance with an exemplary embodiment; and

FIG. 10 is a flow chart for a method for classifying an ophthalmic image for diagnosing an ocular disease or condition, in accordance with an exemplary embodiment.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

For the sake of brevity, conventional techniques and components for mathematical processes, transforms, image manipulation, and/or the like may not be described in detail herein. Furthermore, the connecting lines shown in various figures contained herein are intended to represent exemplary functional relationships and/or physical couplings between various elements. It should be noted that many alternative or additional functional relationships or physical connections may be present in exemplary methods and systems for imaging and/or components thereof.

Non-mydriatic retinal color fundus photography (CFP) is widely and routinely used to diagnose ocular diseases. Non-mydriatic CFP can be desirable because it does not require pupillary dilation; however, it is prone to poor image quality. Automated analyses are being developed for point-of-care disease screening (e.g., diabetic retinopathy (DR), age-related macular degeneration, inherited retinal conditions, and retinopathy of prematurity) based on non-mydriatic CFP obtained in a primary care provider's office. Furthermore, research has been conducted to unlock the CFP potential to screen for neurodegenerative disorders such as Alzheimer's disease (AD). Both human and computer-aided analysis methods prefer operating on high-quality retinal CFPs. Non-mydriatic retinal CFP is patient and provider-friendly, but it is also prone to noise, e.g., shade artifacts and blurring due to light transmission disturbance, defocusing, abnormal pupils, or suboptimal human operations, resulting in low-quality CFPs. CFP degradation by obscuration of blood vessels, and missing or artifactual new lesions, leads to inaccurate diagnostic interpretation. Enhancing low-quality retinal CFPs into high-quality counterparts is of key importance for many downstream tasks, e.g., diabetic retinopathy (DR), blood vessel segmentation, DR lesion segmentation, DR diagnostic stratification, accurate AD screening, and the like. Accordingly, improved approaches are desirable.

In clinical settings, medical providers often have difficulty collecting paired low-high quality CFPs. Most denoising frameworks are based on supervised learning and require a pair of data (low quality and high quality). Additionally, the diagnostic interpretation requires high-quality CFPs, but non-mydriatic CFPs are prone to noise and artifacts, leading to inaccurate and sometimes hazardous diagnoses.

Principles of the present disclosure address these issues by eliminating the need for paired data while maximizing the preservation of lesion features in fundus images.

In accordance with various exemplary embodiments, the present disclosure contemplates an integrated unsupervised end-to-end image enhancement framework based on optimal transport (OT) and regularization by denoising methods. The novel OT formulation maximally preserves structural consistency (e.g., lesions, vessel structures, optical discs) between enhanced and low-quality images to prevent over-tampering important structures. To further improve the flexibility and robustness of retinal images from different distributions and enhance the applicability in real-world clinical practice where insufficient data is available to train the model, exemplary approaches refine the enhanced images by a disclosed regularization by enhancing (RE), a variant of regularization by denoising (RED) method, whose priors were learned by the OT-guided network.

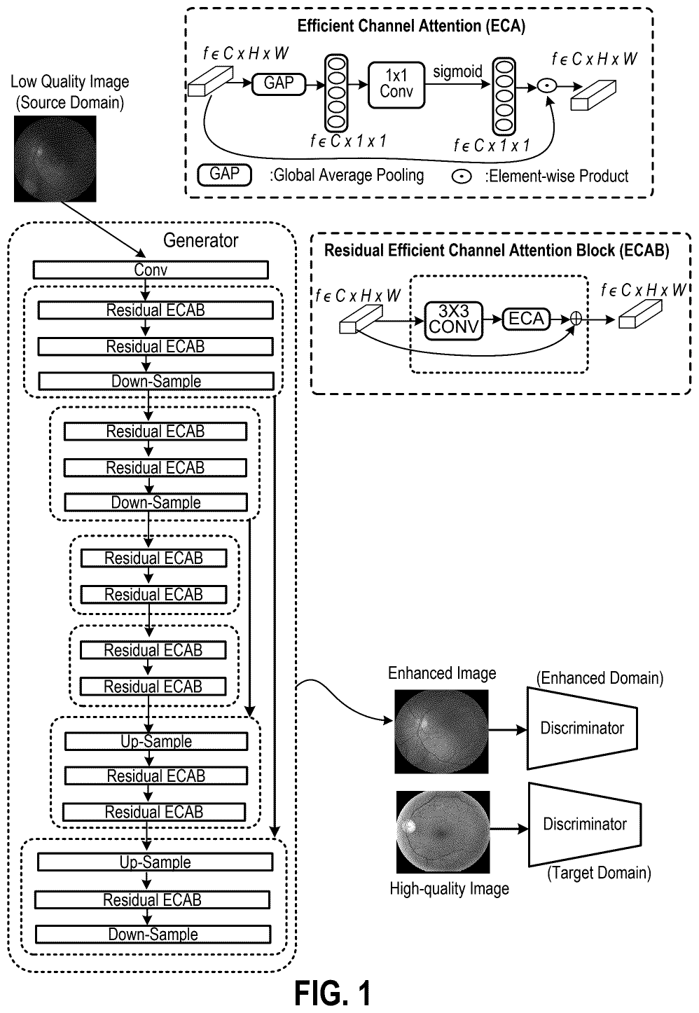

In an exemplary embodiment, and with reference now to FIGS. 1-3, a disclosed processing pipeline comprises a two-stage framework for retinal CFP enhancement. The first stage starts with deriving an Optimal Transport (OT) guided generative adversarial network to translate low-quality images into high-quality images. To do so, domain and identity transport are utilized to enforce the consistency between low-quality and high-quality image domains. To further enhance the preservation of low-level semantics in retinal fundus images (e.g., optic discs, blood vessels, DR lesions such as exudates, and microaneurysm), maximally information-preserving consistency mechanisms may be utilized by taking advantage of multi-scale structural similarity loss and U-shape neural network architecture.

In the second stage, an exemplary system utilizes an off-the-shield framework to address the applicability and robustness of the OT-guided enhancing network in real-world clinical practice where images come from different distribution/institutes, with insufficient data available for end-to-end training called regularization by enhancing. The exemplary approach extends the denoiser-centric view of regularization by denoising (RED) to a more generic version that leveraged the image prior learned from the proposed OT-guided enhancing network.

An exemplary final integrated framework performs iterative updating for each testing image based on the disclosed regularization by enhancing the framework and learning prior from the disclosed OT-guided enhancing network until convergence.

As compared to prior approaches, principles of the present disclosure contemplate (1) a novel OT-guided GAN-based unsupervised end-to-end retinal image enhancement training scheme, where a maximal information-preserving consistency mechanism is adopted to prevent lesion and structure over-tampering; and (2) an RE module introduced to refine the OT module's output, improving the system's flexibility, robustness, and applicability. Exemplary embodiments are believed to be the first of its kind to bridge the gap between OT-guided generative models and model-based enhancement frameworks. It is a general approach, adaptable to other structure-preserving medical image enhancement research.

Moreover, as compared to prior approaches, exemplary systems and methods have the following advantages compared to current alternatives: (1) The unsupervised unpair training method solves the problem of hard collecting a pair of high-quality and correspondingly low-quality. (2) Maximal lesion information preserving. (3) This framework integrated an OT-guided GAN-based enhancing network with the RE module. It achieved promising results on three datasets, surpassing or on par with SOTA unsupervised and supervised methods. (4) This unsupervised enhancement method may also be used in other eye disease studies to improve image quality and help doctors aid in diagnosis.

### Optimal Transport Guided Unsupervised Learning for Enhancing Low-Quality Retinal Images

The process of mapping low-quality retinal images to their high-quality counterparts, where the domain consisting of enhanced images is defined herein as enhanced domain, can be modeled as an end-to-end image-to-image translation task. Various examples leverage generative adversarial networks (GANs) where the adversarial training progressively leads to the photo-realistic renderings. The key idea is to map a source domain y to a target domain x, which maps the source distribution to a target distribution Py→ [symbol] x. This mapping becomes more challenging when the input and target are unpaired because no direct ground-truth data are available. To reduce the searching space of such mappings, a task-specific regularization can be utilized, while the CycleGAN generalizes them as a cycle consistency. However, as a general image generation model, CycleGAN has several drawbacks when applied to retinal image generation. First, it is burdened with its expensive computation with concurrent training of a pair of generators and discriminators. Secondly, it may lose lesions and introduces non-existing vessels. In one example, an optimal transport GAN for unsupervised natural image denoising led to the destruction of vessel and lesion structures. Different from natural images with additive noises, the degradation of retinal images is more complicated and, therefore, more challenging to model.

Disclosed herein is an alternative view of image-to-image translation with a standpoint of optimal transport theory to enforce the consistency between the enhanced domain and the target domain (high-quality images) while preserving the information between the source and target domains to prevent lesion tampering and the generation of unrealistic non-existing blood vessels. In various examples, contributions of optimal transport guided unsupervised learning for enhancing low-quality retinal images of the present disclosure can be summarized in three aspects: (1) An optimal transport-guided domain consistency that ensures the consistency between the enhanced domain and the target domain; (2) A unified GAN-based unsupervised retinal image enhancement training scheme is introduced; and (3) To mitigate the inconsistency of vessels, optic disc, and lesions between before and after enhancements, a maximally information-preserving consistency loss in conjunction with a data resampling mechanism is utilized.

### Methods

A disclosed method consists of three modules: (1) optimal transport-guided domain consistency, (2) maximally information preserving consistency, and (3) refined data resampling for lesion consistency. The entire framework of a disclosed method is shown in FIG. 1.

### Optimal Transport Guided Domain Consistency

Let μ˜ [symbol] x and v˜ [symbol] y denote two probability measures on the input and target probability spaces, respectively. The Monge's optimal transport map G:y→x that minimizes the cost of transporting v to μ is given by

$$G *= {inf}_{f : μ ( X ) = v ( {G}^{- 1} ( X ) )} {\int }_{𝒴} {ℒ}_{c} ( Y , G ( Y ) ) dv ( Y ) \qquad ( Eq . 1 )$$

where [symbol] :Y×X→[0, +∞] denotes the cost function. By parameterizing the optimal transport map as a neural network G_{θ}, Equation 1 can be discretized to

$$θ *= {arg min}_{θ : {ℙ}_{𝒳} = {ℙ}_{{G}_{θ} ( Y )}} {𝔼}_{Y ∼ {ℙ}_{𝒴}} [ {ℒ}_{c} ( Y , {G}_{θ} ( Y ) ) ] . \qquad ( Eq . 2 )$$

It is worth noticing that Equation 2 yields an unsupervised training scheme where the minimization is decoupled with the high-quality images x, and the constraint can be achieved by unpaired adversarial training. The constrained optimization can be further relaxed to an unconstrained optimization by applying Lagrange multiplier to Equation 2 yielding

$$θ *= {arg min}_{θ} {𝔼}_{Y ∼ {ℙ}_{𝒴}} [ {ℒ}_{c} ( Y , {G}_{θ} ( Y ) ) ] + λ d ( {ℙ}_{𝒳} , {ℙ}_{{G}_{θ} ( Y )} ) \qquad ( Eq . 3 )$$

where d measures the divergence between two distributions with d(:,:)≤0. To take advantage of GANs, the divergence constraint d(:,:)≤0 is achieved adversarially by optimizing the Wasserstein-1 distance given by

$${𝒲}_{1} ( {ℙ}_{𝒳} , {ℙ}_{{G}_{θ} ( Y )} ) = {sup}_{{‖ {D}_{w} ‖}_{L} \leq 1} {𝔼}_{X ∼ {ℙ}_{𝒳}} [ {D}_{w} ( X ) ] - {𝔼}_{Y ∼ {ℙ}_{𝒴}} [ {D}_{w} ( {G}_{θ} ( Y ) ) ] \qquad ( Eq . 4 )$$

where D_{w} denotes the discriminator parameterized by w with a 1-Lipschitz constraint which is approximated by Gradient Penalty in the experiments.

Equation 3 implies the consistency between the source and a target domain is traded off by the consistency between the target domain and the enhanced domain, which matches initial expectations that the distribution of the enhanced low-quality images should align with that of the high-quality images while having the same underlying structures as the low-quality images. Low-quality enhancement could be fully achieved by training a GAN with generator G_{θ} and discriminator D_{w} via optimizing the following objective function:

$${max}_{{G}_{θ}} {min}_{{D}_{w}} {𝔼}_{Y ∼ {ℙ}_{𝒴}} [ {ℒ}_{c} ( Y , {G}_{θ} ( Y ) ) ] + λ {𝒲}_{1} ( {ℙ}_{𝒳} , {ℙ}_{{G}_{θ} ( Y )} ) \qquad ( Eq . 5 )$$

### Maximal Information Consistency

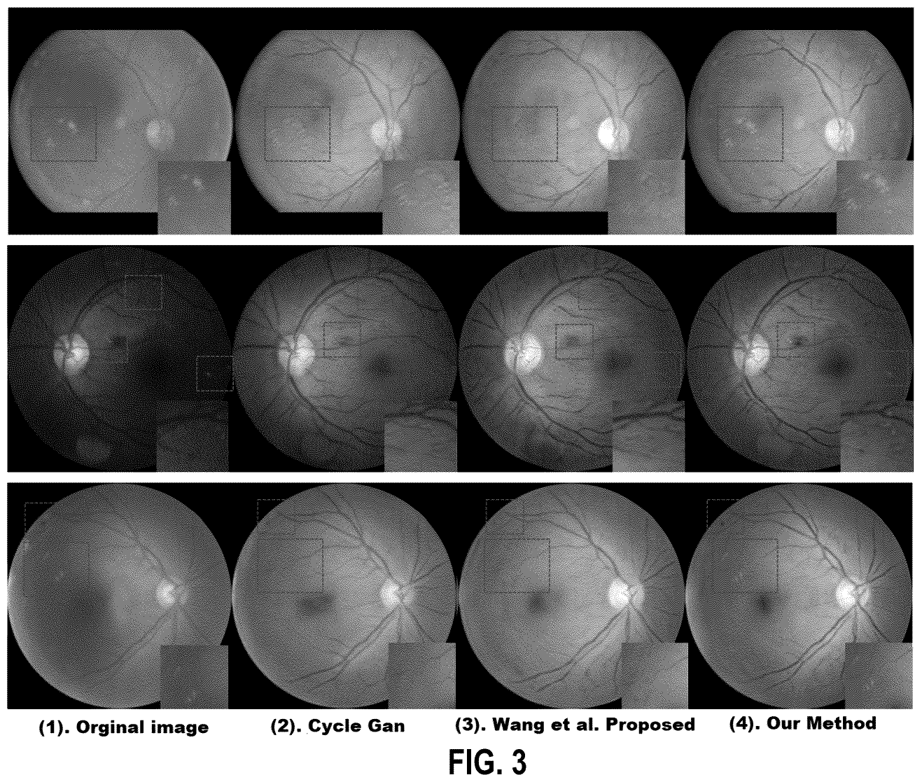

Next, it will be explained how to choose the loss function Le to enforce data consistency for this task. When [symbol] is convex, the strong duality between Equation 3 and Equation 2 holds, meaning they achieve identical optimality. Conventionally, the L₁ or L₂ norm is a common convex choice to enforce the data consistency, where L₁ norm leads to the optimal median while L₂ norm leads to the optimal mean from a statistical point of view. But either L₁ or L₂ norm will result in blurring in rendered images because of the smoothness of sharp edges and loss of high-frequency local structures, which is undesired. From early experiments with CycleGAN using L₁ norm as a consistency loss, pathologically meaningful structures were observed, particularly lesions in diabetic retinopathy, were lost in the enhancement, as shown in FIG. 3. To preserve more local structures, particularly those that are pathologically meaningful, Multi-Scale Structural Similarity Index Measure SSIMMS is chosen as the consistency loss, which is given by

$${ℒ}_{c} ( Y , {G}_{θ} ( Y ) ) = 1 - {SSIM}_{MS} ( Y , {G}_{θ} ( Y ) ) , \qquad ( Eq . 6 )$$

where G_{θ}(Y) is the rendered high-quality images. The SSIM consistency loss is locally quasi-convex minimizing the duality gap between Equations 3 and 2 while maximizing the mutual information between the source domain and enhanced domain.

The enhanced high-quality images from their low-quality counterparts, in general, have the same underlying structures, e.g., blood vessels, lesions, optical disks, etc. With this observation, a specific U-shape generator is provided, enabling the low-level semantic information flow from low-quality images to their high-quality enhancements. To further augment the performance of lesion preservation, an efficient channel attention block is added to each residual block in the generator. Because different types of diabetic lesions have different sensitivity in different image channels.

### Lesion Consistency Via Refined Resampling

Despite the maximal mutual information consistency loss, facilitating lesion consistency between the low-quality images and their enhancements is still a challenging task due to the variation in the occurrence of lesions. In diabetic retinopathy, images at different diabetic retinopathy levels consist of different types of lesions. Even images at the same diabetic retinopathy level are likely to consist of different lesions, e.g., in proliferative diabetic retinopathy, the lesion may include hard exudates, hemorrhages, and microaneurysms. To mitigate this issue, prior knowledge of the lesions is taken into account for sampling input-target pairs by ensuring the input and target are at the same diabetic retinopathy level. Not only does doing so guide the lesion consistency between the low quality images and their enhancements, but it also ensures that the distribution of lesions in the enhanced images is close to that in the low-quality images.

### Experiments

An exemplary method was evaluated through two major experiments: No-Reference Quality Evaluation and Full-Reference Quality on the EyeQ dataset. The main comparisons are with the popular unsupervised image-to-image translation and noise reduction adversarial generation models, including (1) CycleGAN, and (2) optimal transport-based method (OTTGAN).

### Dataset

The EyeQ dataset consists of 9239 training images and 11362 testing images. The original dataset is manually labeled into three quality levels: good, usable, and reject. The goal is to convert all reject images to high-quality images (good). All models were trained on the official training set: 6342 good images and 1544 reject images and evaluated on the official testing dataset: 5966 good and 2195 reject images.

### Implementation Details

An exemplary method was implemented in PyTorch. For training and testing, all images were center-cropped and resized to a size of 256×256. Data augmentations, including random horizontal flips, vertical flips, random crops, and random rotations, were performed during training to prevent over-fitting. The methods compared with the present method were also trained with their official implementations for comparison. To maintain the fairness of all comparisons, all models were trained with an RMSprop optimizer for 200 epochs with an initial learning rate of 0.0001 for the discriminator and 0.00005 for the generator. The learning rate decayed by 10 every 100 epochs. The optimal λ which yielded results for this paper was 40.

### Evaluation Metrics

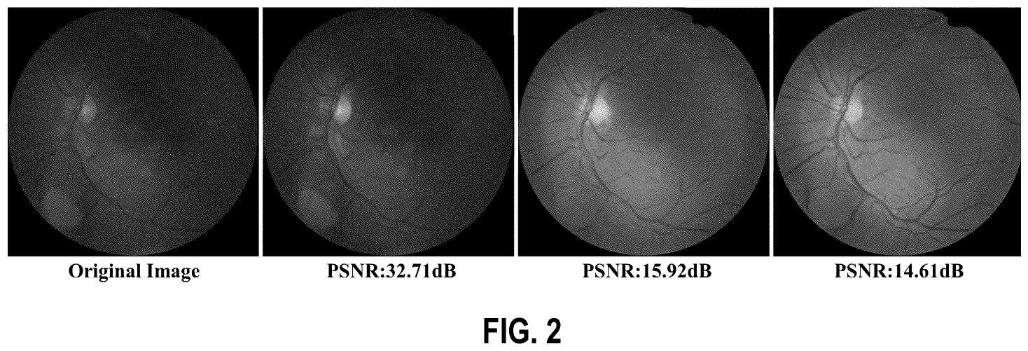

No-Reference Quality Assessment. Evaluating the quality of the enhancements without having access to the ground-truth high-quality images is challenging. The peak signal-to-noise ratio (PSNR) and structural similarity index measurement (SSIM) can be measured between the enhancements and the input low-quality images. As shown in FIG. 2, the PSNR between the low-quality images and their enhancements do not necessarily yield a high PSNR and SSIM. Two evaluation metrics were conducted to quantitatively assess the performance of the enhancements without knowing their ground-truth high-quality counterparts.

The first no-reference quality evaluation metric is the Converted Ratio (CR) which is defined as the percentage of high-quality images among the enhancements. A ResNet50 with efficient channel attention was trained on the EyeQ dataset with three labels (high quality, usable, and reject) to predict the quality of retinal images. The trained CR evaluation model achieved a Cohen's kappa coefficient of 0.918 and an AU-ROC of 0.976 on the EyeQ testing set.

Secondly, a task-specific evaluation is performed on Diabetic Retinopathy prediction indicated by Classification Accuracy, Cohen's kappa coefficient (kappa), and Area under the receiver operating characteristic curve (AU-ROC). Training was performed on the enhanced images produced by three models, which were used for model evaluation based on the EyeQ testing set. As shown in Table 1, the disclosed method outperformed the other two competitors in all evaluation metrics.

Perceptually, CycleGAN and the OTT-GAN change the structure of vessels and smooth lesions to some extent, as shown in FIG. 3. The disclosed method maintained the consistency of local structures of optic discs, vessels, and particularly lesions between low-quality images and their enhancements compared to the other two competitors.

TABLE 1
Comparison No-Reference Evaluation Metrics.

| Method | CR | Accuracy | Kappa | AUC |
|---|---|---|---|---|
| CycleGAN | 0.2199 | 0.7148 | 0.5378 | 0.9083 |
| Wang et al. | 0.1835 | 0.6996 | 0.5105 | 0.8995 |
| Ours | 0.2404 | 0.7450 | 0.6349 | 0.9255 |

Full-Reference Quality Assessment. The 1400 high-quality images randomly selected from the EyeQ test dataset were intentionally degraded to simulate light interference, image blurring, and image artifacts.

The performance of enhancement was evaluated with a PSNR and SSIM between the degraded low-quality images and their high-quality counterparts. The disclosed method demonstrated superiority over the other two competitors statistically in terms of PSNR and SSIM, as shown in Table 2.

TABLE 2
Comparison Full-Reference Evaluation Metrics.

| Method | PSNR | SSIM |
|---|---|---|
| Degration Images | 17.2966 | 0.7399 |
| CycleGAN | 18.6820 | 0.7370 |
| Wang et al. | 18.7705 | 0.7335 |
| Ours | 19.6757 | 0.7631 |

In summary, the above-mentioned work proposed an efficient and effective GAN-based unpaired image-to-image translation framework to tackle the blind enhancement of poor-quality retinal fundus images built on the optimal transport theory. To further enforce the consistency of local semantics (optical discs, vessel structures, lesions, etc) before and after the enhancement, a maximal mutual information consistency mechanism was disclosed with a consistency loss and a lesion resampling strategy, in conjunction with a specific network design. Perceptual and quantitative no-reference and full-reference assessments on the EyeQ dataset showed the superiority of the disclosed method over two other methods. Aside from the PSNR and SSIM image-quality evaluation metrics, the effectiveness of the disclosed method on task-specific evaluation of diabetic retinopathy was demonstrated, suggesting the potential of this work in assisting real-world clinical diagnoses.

### OTRE: Where Optimal Transport Guided Unpaired Image-to-Image Translation Meets Regularization by Enhancing

In various embodiments, image enhancement methods of the present disclosure leverage the Optimal Transport (OT) theory to propose an unpaired image-to-image translation scheme for mapping low-quality retinal CFPs to high-quality counterparts. Furthermore, to improve the flexibility, robustness, and applicability of the image enhancement pipeline in the clinical practice, aspects of the present disclosure generalize a state-of-the-art model-based image reconstruction method, regularization by denoising, by plugging in priors learned by the OT-guided image-to-image translation network. This image reconstruction method is referred to herein as regularization by enhancing (RE). The integrated framework, OTRE, was validated on three publicly available retinal image datasets by assessing the quality after enhancement and their performance on various downstream tasks, including diabetic retinopathy grading, vessel segmentation, and diabetic lesion segmentation. The experimental results demonstrated the superiority of the disclosed framework over some state-of-the-art unsupervised competitors and a state-of-the-art supervised method.

Having provided an overview of the disclosed image enhancement methods and aspects thereof, example image enhancement methods, including an OT-guided GAN-based enhancing network with an RE module, are described in greater detail in the following paragraphs.

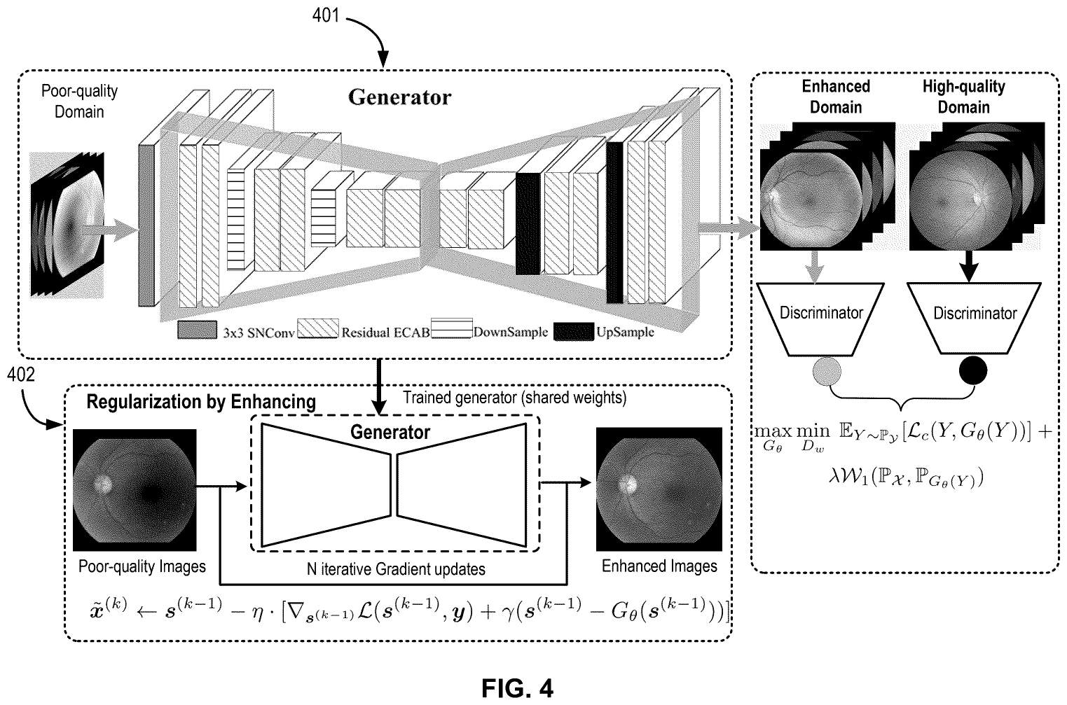

Restoring clean images x˜X from their corruptions y˜Y can be formulated as a variational regularization in the Bayesian framework

$$\hat{x} = {arg min}_{x} f ( x ) + R ( x ) , \qquad ( Eq . 7 )$$

where f is the data fidelity measuring the consistency between the restoration and the corrupted data and R is the regularization/prior term. The modern deep learning-based image restoration seeks to train an end-to-end regressor by minimizing the empirical risk E_{x,y}[L(f_{θ}(y), x)], where f_{θ} is a neural network parameterized by θ, and L is the loss function. Recent advances show that plugging a learned image regressor into the model-based restoration framework boosts its performance. This work unified the model-based regularization and the learned image restoration regressor to provide a flexible, robust framework for enhancing low-quality retinal fundus images. In various examples, the framework includes two main modules as shown in FIG. 4: 1) a first module 401 including an OT-guided unsupervised GAN learning scheme serving as a regressor to enhance low-quality images to pursue f_{θ} in Equation 7, and 8) a second module 402 including an explicit regularization term RE as R(x), refining the trained generator networks obtained in the first module. The two modules are cascaded together. The entire framework is iterated until both modules converge.

### OT-Guided Unpaired Image-to-Image Translation

Let u˜ [symbol] x and v˜ [symbol] y be two probability measures on the target and source probability manifolds, respectively. The Monge's optimal transport problem of transporting masses from domain Y to X (Y→X) can be defined as

$$inf {\int }_{Y} C ( y , T ( y ) ) dv ( y ) \qquad ( Eq . 8 )$$

where C (⋅,⋅) is the cost of transporting y to T(y). The minimal cost among all possible v-mensurable mappings T yields the optimal transport u=T*(v). The transport defined in Equation 8 matches the objective of Image-to-Image translation which seeks an optimal mapping from the source domain to the target domain, which is defined herein as Domain transport. The proposed OT-guided Image-to-Image translation scheme is turned into an optimization problem.

The Image-to-Image translation from a source to a target domain Y→X suggested by the optimal mass transport can be expressed as

$$inf {\int }_{Y} C ( y , T ( y ) ) dv ( y ) , subject to u = {T}^{*} ( v ) \qquad ( Eq . 9 )$$

By further parameterizing the optimal transport map T as a neural network T_{θ}, the Equation 9 can be discretized as

$${min}_{θ} {𝔼}_{y \sim {ℙ}_{Y}} [ C ( y , {T}_{θ} ( y ) ) ] , subject to {ℙ}_{{T}_{θ} ( Y )} = {ℙ}_{X} \qquad ( Eq . 10 )$$

By applying the Lagrange Multiplier, Equation 10 is relaxed to a constrained optimization given by

$${min}_{θ} {𝔼}_{y \sim {ℙ}_{Y}} [ C ( y , {T}_{θ} ( y ) ) ] , + λ d ( {ℙ}_{{T}_{θ} ( Y )} = {ℙ}_{X} ) , \qquad ( Eq . 11 )$$

Likewise, transporting a given measurement in the target domain will also produce another measurement in the target domain X. However, discrepancies between the measurements on the target tend to be undesirable. An Identity cost constraint is introduced to prevent the network from over-learning or generating unexpected measurements. The constraint is utilized for maintaining consistency in the target domain. Adding this term to Equation 11 can be expressed as:

$${min}_{θ} {𝔼}_{y \sim {ℙ}_{Y}} [ C ( y , {T}_{θ} ( y ) ) ] , + {𝔼}_{x \sim {ℙ}_{X}} [ C ( x , {T}_{θ} ( x ) ) ] + λ d ( {ℙ}_{{T}_{θ} ( Y )} , {ℙ}_{X} ) , \qquad ( Eq . 12 )$$

Algorithm 1 OT-Guided Unpaired Image-to-Image Translation.

```
Require: The learning rate η, the batch size m, the gradient penalty weight
  λ, the consistency loss weight α ≤ 1, the identity loss weight β.
Require: Initial discriminator parameters w₀, initial generator parameters θ₀.
  while not converge do
    $Sample a batch of low ‐ quality images y = {\{ {y}_{i} \}}_{i = 1}^{m} ∼ {ℙ}_{Y} with {\{ {g}_{i} \}}_{i = 1}^{m} .$
    $Sample a batch of high ‐ quality images x = {\{ {x}_{i} \}}_{i = 1}^{m} ∼ {ℙ}_{X} with {\{ {g}_{i} \}}_{i = 1}^{m} .$
    for i = 1, . . . , m do
      Sample a random ϵ~U[0, 1].
      \tilde{x}ᵢ ← G_{θ}(yᵢ)
      \hat{x}ᵢ ← ϵxᵢ + (1 − ϵ) \tilde{x}ᵢ
      ${ℒ}_{{D}_{w}} ( i ) ← {D}_{w} ( {\tilde{x}}_{i} ) - {D}_{w} ( {x}_{i} ) + {λ ( {\vert \vert {∇}_{{\hat{x}}_{i}} {D}_{w} ( {\hat{x}}_{i} ) \vert \vert}_{2} - 1 )}_{+}^{2}$
    end for
    $w ← w + η \cdot RMSProp ( w , {∇}_{w} \frac{1}{m} {\sum }_{i = 1}^{m} {ℒ}_{{D}_{w}} ( i ) )$
    ${ℒ}_{{G}_{θ}} ← \frac{1}{m} {\sum }_{i = 1}^{m} - {D}_{w} ( {G}_{θ} ( y ) ) + α {ℒ}_{d} ( y , {G}_{θ} ( y ) ) + β {ℒ}_{idt} ( x , {G}_{θ} ( x ) )$
    θ ← θ − η · RMSProp(θ, ∇_{θ} [symbol] G_{θ})
  end while
```

which is defined as Identity constraint, where d(⋅,⋅) measures the divergence of [symbol] x and [symbol] _{Tθ(Y)}, and λ is a weight parameter. It is noteworthy that the same cost representation is used, but the Identity term is utilized as a constraint in the target domain X and is not related to the optimal transport between the source and target domain.

### Proposition 1

Supposing Wasserstein-1 distance W₁(⋅,⋅) is applied to measure the divergence between [symbol] x and [symbol] _{Tθ}(Y), Equation 12 suggests an adversarial training scheme of unpaired Image-to-Image translation from Y→X, given by

$${max}_{{G}_{θ}} {min}_{{D}_{w}} {𝔼}_{Y} [ {ℒ}_{d} ( y , {G}_{θ} ( y ) ) ] + {𝔼}_{X} [ {ℒ}_{idt} ( x , {G}_{θ} ( x ) ) ] + {λ𝒲}_{1} ( {ℙ}_{X} , {ℙ}_{{G}_{θ} ( Y )} ) {𝒲}_{1} ( {ℙ}_{X} , {ℙ}_{{G}_{θ} ( Y )} ) = {sup}_{{‖ {D}_{w} ‖}_{L} \leq 1} {𝔼}_{X} [ {D}_{w} ( x ) ] - {𝔼}_{Y} [ {D}_{w} ( {G}_{θ} ( y ) ) ] \qquad ( Eq . 13 )$$

where G_{θ} is the generator parameterized by θ, D_{w}, the discriminator, is a 1-Lipschitz function parameterized by w, and [symbol] and [symbol] denotes the domain transport cost and identity constraint cost, respectively. To better preserve the important structure (e.g., lesions), it is ensured that the unpaired input with the matched disease labels gi if there were any, where g denotes the disease type.

For example, regarding the EyeQ dataset: the dataset was designed for a diabetic retinopathy grading classification task. This grading delineates five levels of severity: no retinopathy, mild non-proliferative DR (NPDR), moderate NPDR, severe NPDR, and proliferative DR (PDR). IN various exemplary embodiments where the unpaired enhancing framework training is performed, the same disease grading label image pairs (low-high quality image at same grading level) may be utilized. Moreover, for the same lesion of grading label images, the lesion may be very different, e.g., for a grading 4 retinal image, the lesion may have hard exudates, hemorrhages, microaneurysms, and so on. Accordingly, various exemplary embodiments may utilize a priori knowledge of the lesion for sampling. It will be appreciated that this approach not only guides the lesion reconstruction but also ensures that the reconstructed lesions are in the same distribution as the real ones. Similar principles are seen in various other exemplary retinopathy datasets, where lesion labels, if present, are purposively sampled in pairs of unpaired HIGH-LOW QUALITY IMAGES to select labels of the disease level. It will be appreciated that if lesion labels are not present, it is possible to sample only from the generalized high and low quality. Accordingly, exemplary embodiments may utilize either suitable approach.

The 1-Lipshcitz constraint is approached by the gradient penalty in various examples. The Domain transport and the Identity constraint shares the same cost function, as detailed below.

### Information-Preserving Consistency Mechanism

In various embodiments, there are two main concerns of the proposed OT-guided unpaired image-to-image translation: 1) maintaining the underlying information, e.g., optical discs, lesions, and vessels, consistency before and after the translation; 2) minimizing the duality gap between the primal problem (Equation 10) and the dual problem (Equation 11). Accordingly, the following introduces the information-preserving consistency mechanism centered on addressing these two main concerns.

CycleGAN addresses the first concern by introducing the L₁ norm as the loss function to enforce low-frequency consistency leading us to the optimal median. In addition, a Patch Discriminator is incorporated to capture high-frequency components by enforcing local structural consistency at a patch level. The Patch Discriminator shall specify architecture with a pre-defined receptive field usually resulting in a “shallow” discriminator. Early experiments with CycleGAN, however, indicated that it destroyed the lesion structures and introduced non-existing vessels. Inspired by the Patch Discriminator, a multi-scale structural similarity index measure (SSIM) is used as the consistency loss function [symbol] given by [symbol] (y, G_{θ}(y))=1−SSIMMS (y, G_{θ}(y)). Followed by the CycleGAN, the identity loss [symbol] is also incorporated to make sure that a high-quality input would result in a high-quality enhancement given by [symbol] (x, G_{θ}(x))=1 SSIM_{M S} (x, G_{θ}(x)). A U-Net architecture is also used as the generator to help the low-level semantics flow from the poor-quality domain to the high-quality domain. In an example, the U-net is a convolutional network architecture for fast and precise segmentation of image(s). The following theorem provides a theoretical guarantee to the loss function definition.

### Theorem 1

The Structural Similarity Index Measure is proven to be locally Quasi-Convex which minimizes the duality gap between the primal and dual problem and weak duality holds.

To better balance identity loss, domain loss, and the divergence between [symbol] x and [symbol] G_{θ}(y) the final objective function was rewritten as

$${max}_{{G}_{θ}} {min}_{{D}_{w}} {\sum }_{i = 1}^{n} [ {αℒ}_{d} ( {y}_{i} , {G}_{θ} ( {y}_{i} ) ) + {βℒ}_{idt} ( {x}_{i} , {G}_{θ} ( {x}_{i} ) ) ] + {𝒲}_{1} ( {ℙ}_{X} , {ℙ}_{{G}_{θ} ( Y )} ) , \qquad ( Eq . 14 )$$

where α, β are weight parameters of the domain loss and identity loss, respectively. The algorithm of the OT-guided unpaired image-enhancing training scheme is given by Algorithm 1.

### Regularization by Enhancing

Regularization by Denoising (RED) is a model-based framework that can take advantage of a variety of existing CNN priors without modifying the model's architecture to guide image restoration. The denoiser-centered RED idea is generalized to a more generic one that leverages the image prior learned from the proposed OT-guided enhancing networks. The enhancement is formulated as an image prior to guiding the restoration of any test images whenever there are not enough samples for the end-to-end training. The objective of the proposed regularization by Enhancing (RE) is given by

$$\hat{x} = {arg min}_{x} {𝔼}_{x} [ ℒ ( x , y ) ] + γ R ( x ) with R ( x ) = \frac{1}{2} {x}^{T} ( x - {G}_{θ} ( x ) ) , \qquad ( Eq . 15 )$$

where γ controls the regularization strength, and L denotes the multi-scale structural similarity loss. The gradient of the RE prior has a simple form

$${∇}_{x} R ( x ) = x - {G}_{θ} ( x ) , \qquad ( Eq . 16 )$$

under the condition that G_{θ} is locally homogeneous and has a symmetric Jacobian. The 1-Lipschitz constraint of G_{θ} can further guarantee the passivity of G_{θ} resulting in a convex objective function. The spectral radius of the weight of each convolutional layer in the generator G_{θ} was regularized via spectral normalization to approximate the 1-Lipschitz constraint. In the optimization phase, the accelerated gradient descent was chosen to iteratively approach the optimum. The iterative optimization of the RE is given by Algorithm 2.

Algorithm 2 Regularization by Enhancing.

```
Require: The step size η, regularization strength γ, tolerance tol, Generator
  G_{θ}
Require: Initial \tilde{x}⁽⁰⁾, s⁽⁰⁾ = \tilde{x}⁽⁰⁾, t⁽⁰⁾ = 1
  while not converge do
    ${t}^{( k )} = \frac{1}{2} ( 1 + \sqrt{1 + 4 {( {t}^{( k - 1 )} )}^{2} )}$
    $Der ( {s}^{( k - 1 )} ) = {∇}_{{s}^{( k - 1 )}} ℒ ( {s}^{( k - 1 )} , y ) + γ ( {s}^{( k - 1 )} - {G}_{θ} ( {s}^{( k - 1 )} ) )$
    \tilde{x}^{(k)} ← s^{(k-1)} − η · Der(s^{(k-1)})
    ${s}^{( k )} ← {\tilde{x}}^{( k )} + \frac{{t}^{( k - 1 ) - 1}}{{t}^{( k )}} ( {\tilde{x}}^{( k )} - {\tilde{x}}^{( k - 1 )} )$
    if ||\tilde{x}^{(k)} − \tilde{x}^{(k-1)}|| ≤ tol · ||\tilde{x}^{(k-1)}|| then
      break
    end if
  end while
```

### Experimental Results

Extensive experiments were conducted in scenarios where the ground-truth clean images are available (full-reference assessment) and unavailable (no-reference assessment). Three downstream tasks including DR grading, vessel segmentation, and lesion segmentation were studied to further evaluate the performance of the disclosed method. Visual inspection was conducted by human ophthalmologists to evaluate the performance of no-reference assessment. The vanilla ResNet-50 and U-Net were used to train and test the downstream tasks.

### Datasets

The disclosed method was extensively evaluated on three publicly available retinal CFP datasets: the EyeQ dataset, the DRIVE dataset, and the IDRID dataset. The EyeQ dataset was manually labeled into three quality levels: good, usable, and reject. 7886 training images and 8161 testing images (good & reject) were used in the training and evaluation. The DRIVE dataset evaluated the disclosed method on the vessel segmentation task with 40 subjects. The IDRID dataset containing 81 subjects with pixel-level annotation of microaneurysms (MA), soft exudates (SE), hemorrhages (HE), and hard exudates (EX) were used to evaluate the disclosed method on DR lesion segmentation.

### Experimental Design

For the no-reference assessment, the OT-guided Image-to-Image translation GAN was trained with 7886 training images on the EyeQ dataset by unpaired low-quality images and high-quality images. It was defined as low-quality to high-quality (low2high) model. For the full-reference assessment, the model was trained on the subset of the high-quality EyeQ training dataset with degraded images and unpaired high-quality images. It was defined as degradation to high-quality (deg2high) model. The disease label g in Algorithm 1 was the DR grading label from the EyeQ. Data augmentation, including random horizontal/vertical flips, random crops, and random rotations, was performed to prevent over-fitting during training. All models were trained with the RMSprop optimizer for 200 epochs with an initial learning rate of 1×10⁻⁴ for the discriminator and 5×10⁻⁵ for the generator with a decay of 10 by every 100 epochs. The optimal hyperparameters were α=60, β=20 for both low2high and deg2high models. In the testing phase, the optimal hyperparameter γ was grid-searched within a range from 1×10⁻³ to 1×10⁻⁴ with the number of iterations equal to 400 for all experiments. All methods were implemented in PyTorch.

### No-Reference Quality Assessment

Evaluating the quality of the enhancement without knowing the ground-truth clean images is challenging. It was considered to combine the DR grading task with visual inspection by human experts to assess the performance of the enhancement. The DR grading task can be viewed as a criterion to judge whether lesion information is preserved after the enhancement. A ResNet-50 model was trained on high-quality images following the experimental setup and evaluated by the low-quality images and their enhancements from different methods. The performance of the enhancement will be indicated by the classification accuracy, Area under Receiver Operating Characteristic Curve (AU-ROC), and Cohen's Kappa Coefficient (kappa).

TABLE 3
Evaluation metrics of the DR grading task with enhancements
from different methods in the Lesion structure changed
ratio (LCR), background-color changed ratio (BCR),
and generated extra structure ratio (GESR).

| | DR. Grading | | | Experts Evaluation | | |
|---|---|---|---|---|---|---|
| Method | Accuracy | Kappa | ROC | LCR | BCR | GESR |
| CycleGAN | 0.7148 | 0.5378 | 0.9083 | 0.449 | 0.0 | 0.347 |
| OTTGAN | 0.6996 | 0.5105 | 0.8995 | 0.429 | 0.102 | 0.490 |
| OTRE | 0.7767 | 0.6814 | 0.9408 | 0.020 | 0.040 | 0.326 |

For visual inspection by human experts, 50 low-quality images were randomly chosen and processed by different enhancement methods. Visual inspection was done to measure 1) the ratio of changing lesion structure (LCR), 2) the ratio of changing the main background color (BCR), and 3) the ratio of generating non-existing structures (GESR). For the fairness of the experiments, the assessment was first conducted by three volunteers who were pretrained with the designed protocol and then finalized by the ophthalmologist.

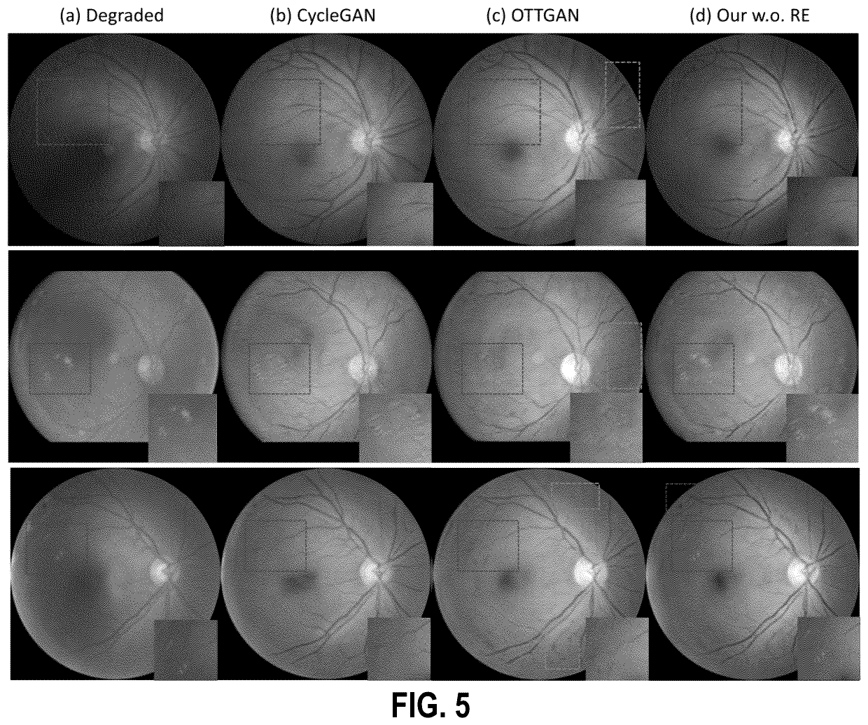

FIG. 5 illustrates results from different unsupervised enhancement methods. The highlight boxes in FIG. 5 denote the structure of the lesion and vessel. It can be observed that all other methods changed the structure of the lesion or vessel. All methods can enhance image quality. However, the disclosed method can better maintain the lesion and vessel structure while reducing the noise. Two experiments were introduced to verify that the disclosed method can preserve the maximal information (see Table 3). First, DR grading algorithm was applied to the enhanced images (low2high) and evaluate their grading accuracy. As shown in Table 3, the OTRE outperformed other methods in all three measures, especially by more than 20% in the kappa measure. The human expert inspection also verified that the disclosed method could maximize information preservation. The disclosed method performed best in LCR and GESR, with a dramatic improvement in LCR, even over 40% improvement over the other two methods.

### Full-Reference Quality Assessment

For full-reference assessment, high-quality images were degraded following the degradation model to synthesize the low-quality images for each dataset. The training dataset consisted of 6500 high-quality images selected from the EyeQ training dataset and other 6500 synthesized low-quality images degraded from non-overlapping 6500 high-quality images from the EyeQ training dataset. The testing dataset was made up of 500 images from the EyeQ testing dataset, the entire DRIVE dataset, and the entire IDRID dataset. The commonly used Peak-Signal-to-Noise Ratio (PSNR) and Structural Similarity Index Measure (SSIM) were used to evaluate the quality of the enhanced low-quality images. To further validate the disclosed method on the downstream tasks, the performance of the disclosed method was evaluated on the blood vessel segmentation and DR lesion segmentation tasks.

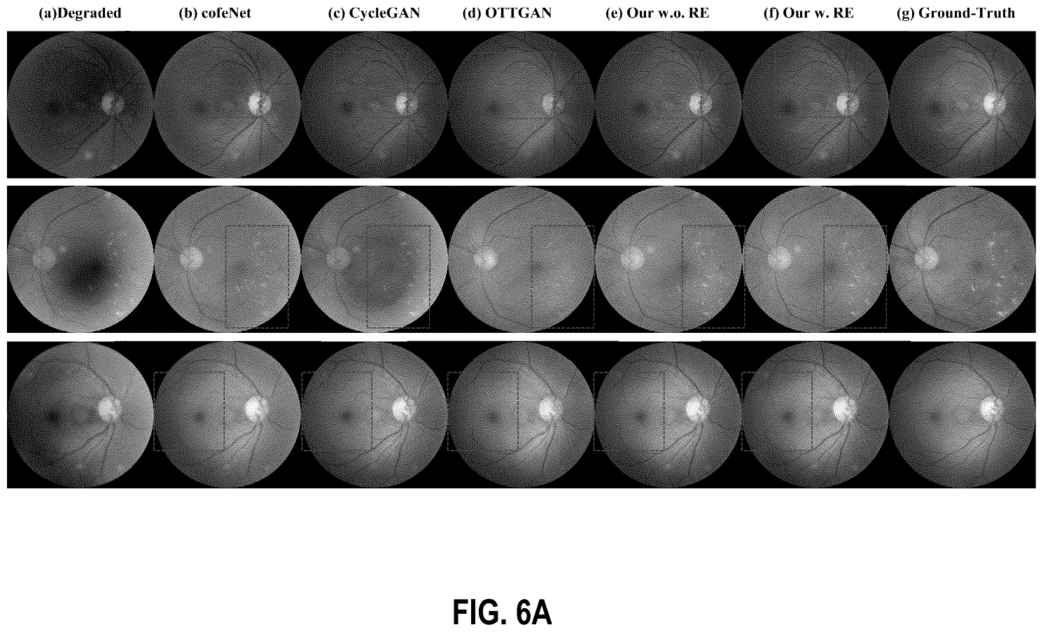

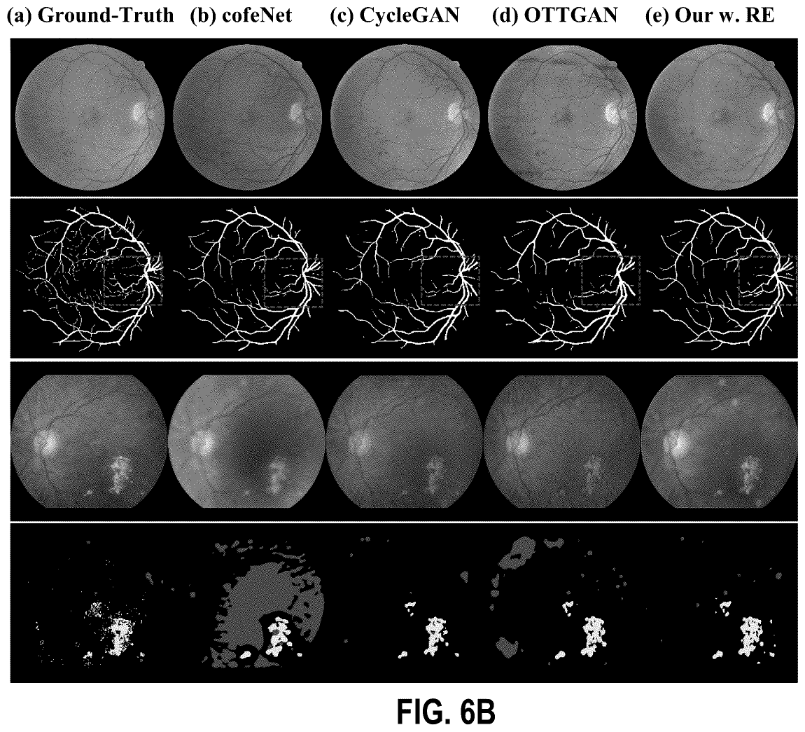

FIG. 6A illustrates deg2high model evaluation based on the full-reference experiment. The highlighted blocks show the comparison of structure preserving and extra structure generation. Visually, the image enhancement is good, but the PSNR is not high, so further downstream task evaluation is essential.

FIG. 6B illustrates a visualization of downstream segmentation tasks. The highlighted blocks denote the comparison of fine vessel bifurcation segmentation results.

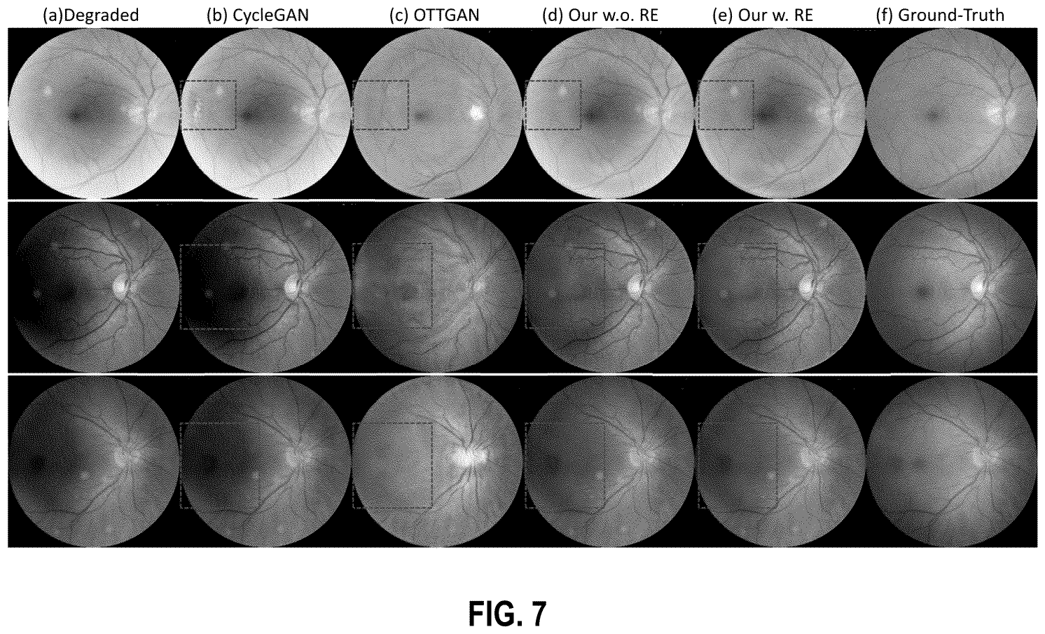

First, the consistency of the enhanced images and their high-quality counterparts was evaluated. Two different experiments were performed by applying both no-reference trained models (Sec. 3.3, low2high) and full-reference trained models (deg2high). It was also tested whether the inclusion of RE module improved the results. FIG. 7 and FIG. 6A show image examples and Table 4 reports the numerical results. As shown in Table 4, except for EyeQ's SSIM measure, the OTRE outperformed all other supervised and unsupervised methods in three different datasets, and the PSNR achieved respectively the highest 24.63, 22.81, and 22.05. Remarkably, the OTRE beat the SOTA supervised method (cofe-Net) given that the cofe-Net was trained with paired images, but the OTRE was not. Interestingly, the method no-reference trained model (low2high) achieved competitive results for the unseen degradation noises. It was also learned that the inclusion of RE module gained improved performance. FIG. 2(B), FIG. 3(A) also provided stronger support of effectiveness that the method preserved the structure and achieved better noise reduction.

TABLE 4
Result comparison of unsupervised methods when trained with the no-reference
training data (Sec. 3.3, low2high) and full-reference training data (deg2high)
on the current degrading testing dataset. The OTRE frameworks with/without
RE module were investigated on both datasets. The supervised method (coef-
Net) was trained/evaluated with the degrading dataset only.

| | | EyeQ | | DRIVE | | IDRID | |
|---|---|---|---|---|---|---|---|
| | Method | PSNR | SSIM | PSNR | SSIM | PSNR | SSIM |
| Supervised | cofe-Net | 23.11 | 0.910 | 21.87 | 0.767 | 20.25 | 0.825 |
| | CycleGAN | 18.57 | 0.836 | 18.78 | 0.705 | 19.13 | 0.799 |
| | (low2high) | | | | | | |
| | CycleGAN | 22.75 | 0.895 | 21.92 | 0.766 | 21.56 | 0.868 |
| | (deg2high) | | | | | | |
| | OTTGAN | 18.93 | 0.859 | 19.20 | 0.723 | 19.70 | 0,828 |
| | (low2high) | | | | | | |
| | OTTGAN | 23.60 | 0.804 | 21.61 | 0.750 | 21.93 | 0.830 |
| | (deg2high) | | | | | | |
| Unsupervised | OTRE without RE | 20.39 | 0.878 | 20.03 | 0.733 | 20.50 | 0.837 |
| | (low2high) | | | | | | |
| | OTRE with RE | 21.08 | 0.880 | 20.61 | 0.740 | 20.55 | 0.836 |
| | (low2high) | | | | | | |
| | OTRE without RE | 24.29 | 0.906 | 22.40 | 0.772 | 21.51 | 0.860 |
| | (deg2high) | | | | | | |
| | OTRE with RE | 24.63 | 0.905 | 22.81 | 0.794 | 22.05 | 0.862 |
| | (deg2high) | | | | | | |

TABLE 5
Result comparison of the segmentation of blood vessels on the DRIVE cohort
and diabetic lesions (EX and HE) on the IDRID dataset. The OTRE compared
favorably to other supervised and unsupervised methods. (ROC: Area under
Receiver Operating Characteristic Curve, PR: Area under the Precision-
Recall, F1: F1 score, SE: Sensitivity, SP: Specificity).

| | Vessel Segmentation | | | | | EX | | | HE | | |
|---|---|---|---|---|---|---|---|---|---|---|---|
| Method | ROC | PR | F1 | SE | SP | ROC | PR | F1 | ROC | PR | F1 |
| cofe-Net | 0.923 | 0.787 | 0.714 | 0.644 | 0.977 | 0.926 | 0.442 | 0.469 | 0.807 | 0.103 | 0.090 |
| CycleGAN | 0.910 | 0.762 | 0.696 | 0.622 | 0.975 | 0.900 | 0.474 | 0.347 | 0.845 | 0.155 | 0.141 |
| OTTGAN | 0.900 | 0.739 | 0.667 | 0.581 | 0.976 | 0.912 | 0.507 | 0.512 | 0.855 | 0.107 | 0.145 |
| OTRE | 0.927 | 0.796 | 0.726 | 0.672 | 0.975 | 0.934 | 0.529 | 0.441 | 0.894 | 0.233 | 0.273 |

To further confirm the superiority of the method, two downstream segmentation tasks were studied using the ground-truth data from DRIVE and IDRID datasets. Since the training and testing of the segmentation task were based entirely on enhanced images, without adding any preprocessing and additional tricks, in the lesion segmentation task, only large blocks of lesions were considered which were easy to train, such as EX and HE. As shown in Table 5, the OTRE method achieved excellent results in three segmentation tasks. It achieved the highest ROC and PR results in all segmentation results, 2 out of 3 bests results in F1 measure. From some image examples shown in FIG. 3B, it is easy to see that other methods have the problem of insignificant enhancement performance, resulting in the altered vessel and lesion structures.

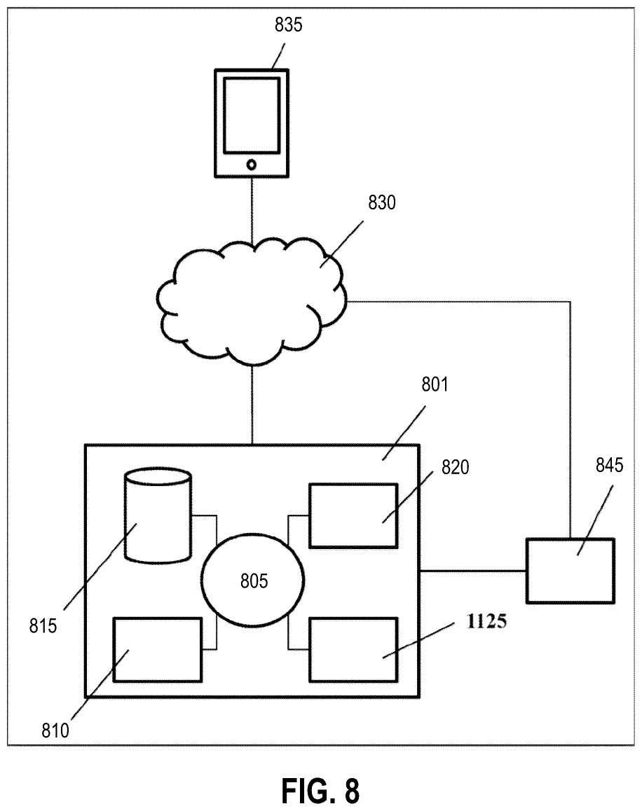

FIG. 8 schematically illustrates a computer control system or platform programmed or otherwise configured to implement the methods provided herein. In some embodiments, the system includes a computer system 801 programmed or otherwise configured to execute executable instructions, such as instructions for performing image analysis and/or image translation. The computer system includes at least one CPU or processor 805. The computer system includes at least one memory or storage unit 810 and/or at least one electronic storage unit 815. In some embodiments, the computer system 801 includes a communication interface 820 (e.g., a network adapter). In some embodiments, computer system 801 may be operatively coupled to a computer network (“network”) 830 by way of the communications interface 820. In some embodiments, an end-user device 835 is used to upload medical data, such as ophthalmic images, general browsing of the database 845, or performance of other tasks. In embodiments, the database 845 is one or more databases separate from computer system 801.

In various embodiments, the memory or storage unit 810 and/or the at least one electronic storage unit 810 includes one or more tangible, non-transitory memories capable of implementing digital or programmatic logic. In various embodiments, for example, the one or more controllers are one or more of a general purpose processor, digital signal processor (DSP), application specific integrated circuit (ASIC), field programmable gate array (FPGA), or other programmable logic device, discrete gate, transistor logic, or discrete hardware components, or any various combinations thereof or the like.

In various embodiments, the memory 810 includes instructions stored thereon that, in response to execution by the processor 805, cause the computer system 801 to perform the methods provided herein.

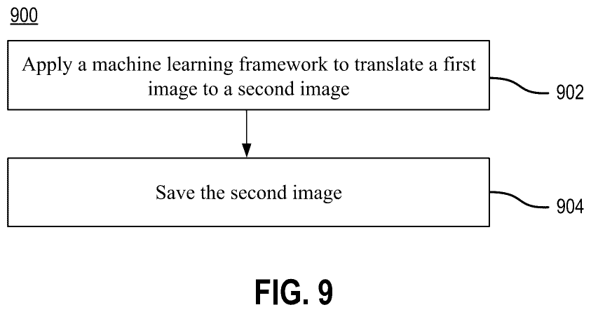

With reference to FIG. 9, a flowchart illustrating a method 900 is provided, in accordance with various embodiments. In various embodiments, the method 900 is a method for image enhancement, for example by translating a first image to a second image (step 902). Step 902 can include applying a machine learning framework to map a source domain to a target domain to thereby translate the first image to the second image. In various embodiments, step 902 includes implementing the framework of FIG. 4. In various embodiments, step 902 includes implementing Algorithm 1 and/or Algorithm 2 provided herein. In various embodiments, step 902 includes implementing Equation 14 as provided herein. In various embodiments, step 902 includes implementing Equation 15 as provided herein. The method 900 can include saving the second image to memory (step 904), for example memory or storage unit 810 and/or the at least one electronic storage unit 810. The method 900 can include identifying structures in the second image (e.g., lesions and/or vessels).

### Machine Learning

In various embodiments, a machine learning method for analyzing medical data, for example, including ophthalmic images (e.g., see FIG. 3) and eye-related data, is disclosed herein. In an exemplary embodiment, the machine learning framework disclosed herein is used to analyze retinal images (e.g., fundus images) to diagnose ophthalmic and/or systemic diseases or conditions. In some embodiments, the prognosis or diagnosis generated according to the systems, methods, and devices described herein includes the detection or diagnosis of an ophthalmic or systemic disease, disorder, or condition. In some embodiments, the prognosis or diagnosis includes assessing the risk or likelihood of an ophthalmic or systemic disease, disorder, or condition. In some embodiments, the prognosis or diagnosis includes a classification or classification of an ophthalmic or systemic disease, disorder or condition. The ophthalmic disease, disorder, or condition can be selected from the group consisting of age-related macular degeneration, diabetic retinopathy, glaucoma, cataract, myopia, retinal vein occlusion, nephropathy, hypertension, and stroke.

In various embodiments, medical imaging is used to perform the predictions or diagnoses. Examples of medical imaging include fundus photographs that can be obtained using a fundus camera that utilizes a dedicated microscope (e.g., an ophthalmoscope). The popularity of fundus photography makes it particularly suitable for rapid and accurate diagnostic screening of ophthalmic and/or systemic diseases. This is especially important in areas where specialists are not readily available, such as rural areas or developing countries/low income environments. Delays in diagnosis and/or treatment can lead to serious consequences that affect health and long-term prognosis. It is recognized in the present disclosure that one solution is to implement a computational decision support algorithm for interpreting medical imaging such as fundus images.

In various aspects, disclosed herein is a method of incorporating machine learning techniques (e.g., deep learning with convolutional neural networks) that demonstrates robust diagnostic capabilities using retinal imaging with a database of retinal images that includes a common database.

Accordingly, in some embodiments, provided herein is an AI transfer learning framework for diagnosing common vision threatening retinal diseases using a dataset of retinal images (e.g., fundus shots) that enables high accuracy diagnoses comparable to human expert performance. In some embodiments, the AI framework classifies images and generates corresponding priorities or labels for the classifications (e.g., “emergency recommendations” or “general recommendations”). In some embodiments, the normal image is labeled “view”. Thus, certain embodiments of the present disclosure utilize the AI framework as a triage system to generate referrals, simulating real-world applications in community environments, primary care and emergency care clinics. These embodiments can facilitate treatments that can improve visual outcome and quality of life by facilitating early diagnosis and detection of disease progression, ultimately affecting a wide range of public health.

In certain aspects, disclosed herein are machine learning frameworks for generating models or classifiers for diagnosing one or more ophthalmic or systemic diseases, disorders, or conditions. These models or classifiers can be implemented in any suitable system or device, for example, a diagnostic kiosk or a portable device, such as a smartphone with an attachable imaging device (e.g., an ophthalmoscope). Non-classifier regression models are also applicable to any of the methods described herein. For example, regression analysis may be performed to generate an output indicative of the severity of an ophthalmologic or systemic disease or disorder.

An example method can include using at least one hardware processor to: receive ophthalmic image data; apply a machine learning classifier to classify the received ophthalmic image data into at least one of a plurality of classifications, the machine learning classifier trained using a domain dataset for an ophthalmic image, the ophthalmic image having been labeled with one or more of the plurality of classifications, wherein the plurality of classifications includes a normal classification and one or more disorder classifications, wherein the one or more disorder classifications include at least one of: age-related macular degeneration (AMD), Diabetic Retinopathy (DR), glaucoma, or Retinal Vein Occlusion (RVO); and provide a report indicative of at least one classification of the received ophthalmic image data.

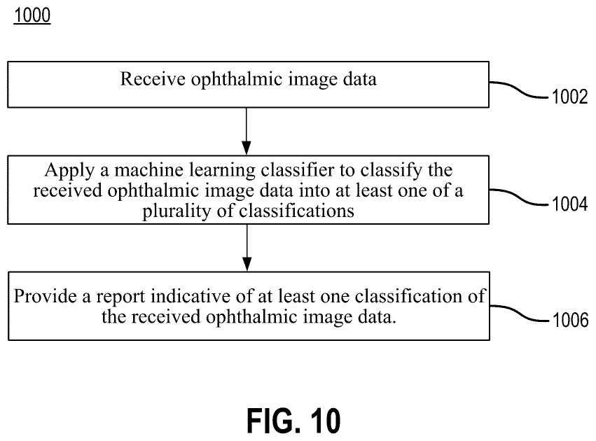

With reference to FIG. 10, a flowchart illustrating a method 1000 is provided, in accordance with various embodiments. In various embodiments, the method 1000 is a method for classifying an ophthalmic image for diagnosing an ocular disease or condition. The method 1000 includes receiving an ophthalmic image data (step 1002). Step 1002 can include receiving an enhanced image generated using the method 900 of FIG. 9 or any of the methods provided herein. The method 1000 further includes applying a machine learning classifier to classify the received ophthalmic image data into at least one of a plurality of classifications (step 1004). Step 1004 can include labeling the ophthalmic image with one or more of the plurality of classifications. The plurality of classifications can include a normal classification and/or one or more disorder classifications, wherein the one or more disorder classifications include at least one of: age-related macular degeneration (AMD), Diabetic Retinopathy (DR), glaucoma, or Retinal Vein Occlusion (RVO). The method 1000 can further include providing a report indicative of at least one classification of the received ophthalmic image data (step 1006).

System program instructions and/or controller instructions may be loaded onto a non-transitory, tangible computer-readable medium having instructions stored thereon that, in response to execution by a controller, cause the controller to perform various operations. The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection. When language similar to “at least one of A, B, or C” or “at least one of A, B, and C” is used in the specification or claims, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. An image enhancement method, comprising:
translating a first image to a second image by applying a machine learning framework to map a source domain to a target domain.

2. The image enhancing method of claim 1, wherein the machine learning framework is a generative adversarial network.

3. The image enhancing method of claim 2, wherein the machine learning framework is an optimal transport-guided unpaired generative adversarial network.

4. The image enhancing method of claim 3, further comprising utilizing the second image to assist in diagnosis of retinopathy.

5. The image enhancing method of claim 4, further comprising classifying the second image into at least one of a plurality of classifications, wherein the plurality of classifications includes a normal classification and one or more disorder classifications, and wherein the one or more disorder classifications includes at least one of: age-related macular degeneration (AMD), Diabetic Retinopathy (DR), glaucoma, or Retinal Vein Occlusion (RVO).

6. The image enhancing method of claim 5, wherein the machine learning framework utilizes the equation:
$${max}_{{G}_{θ}} {min}_{{D}_{w}} {\sum }_{i = 1}^{n} [ {αℒ}_{d} ( {y}_{i} , {G}_{θ} ( {y}_{i} ) ) + {βℒ}_{idt} ( {x}_{i} , {G}_{θ} ( {x}_{i} ) ) ] + {𝒲}_{1} ( {ℙ}_{X} , {ℙ}_{{G}_{θ} ( Y )} ) ,$$
where G_{θ} is a generator parameterized by θ;
D_{w}, a discriminator, is a 1-Lipschitz function parameterized by w;
L_{d} and L_{idt} denotes a domain transport cost and an identity constraint cost, respectively; and
α, β are weight parameters of a domain loss and an identity loss, respectively.

7. The image enhancing method of claim 6, wherein the translating comprises use of an algorithm of the form:
Algorithm 1 OT-Guided Unpaired Image-to-Image Translation.
```
Require: The learning rate η, the batch size m, the gradient penalty weight
  λ, the consistency loss weight α ≤ 1, the identity loss weight β.
Require: Initial discriminator parameters w₀, initial generator parameters θ₀.
  while not converge do
    $Sample a batch of low - quality images y = {\{ {y}_{i} \}}_{i = 1}^{m} ∼ {ℙ}_{Y} with {\{ {g}_{i} \}}_{i = 1}^{m} .$
    $Sample a batch of high - quality images x = {\{ {x}_{i} \}}_{i = 1}^{m} ∼ {ℙ}_{X} with {\{ {g}_{i} \}}_{i = 1}^{m} .$
    for i = 1, . . . , m do
      Sample a random ϵ~U[0, 1].
      \tilde{x}ᵢ ← G_{θ}(yᵢ)
      \hat{x}ᵢ ← ϵxᵢ + (1 − ϵ) \tilde{x}ᵢ
      ${ℒ}_{{D}_{w}} ( i ) ← {D}_{w} ( {\tilde{x}}_{i} ) - {D}_{w} ( {x}_{i} ) + {λ ( {‖ {∇}_{{\hat{x}}_{i}} {D}_{w} ( {\hat{x}}_{i} ) ‖}_{2} - 1 )}_{+}^{2}$
    end for
    $w ← w + η \cdot RMSProp ( w , {∇}_{w} \frac{1}{m} {\sum }_{i = 1}^{m} {ℒ}_{{D}_{w}} ( i ) )$
    ${ℒ}_{{G}_{θ}} ← \frac{1}{m} {\sum }_{i = 1}^{m} - {D}_{w} ( {G}_{θ} ( y ) ) + {αℒ}_{d} ( y , {G}_{θ} ( y ) ) + {βℒ}_{idt} ( x , {G}_{θ} ( x ) )$
    θ ← θ − η · RMSProp(θ, ∇_{θ} [symbol] G_{θ})
  end while
```

8. The image enhancing method of claim 7, wherein the machine learning framework utilizes the equation:
$$\hat{x} = {arg min}_{x} {𝔼}_{x} [ ℒ ( x , y ) ] + γ R ( x ) with R ( x ) = \frac{1}{2} {x}^{T} ( x - {G}_{θ} ( x ) ) ;$$
where γ controls a regularization strength, and L denotes a multi-scale structural similarity loss.

9. The image enhancing method of claim 6, wherein the translating comprises use of an algorithm of the form:
Algorithm 2 Regularization by Enhancing.
```
Require: The step size η, regularization strength γ, tolerance tol, Generator
  G_{θ}
Require: Initial \tilde{x}⁽⁰⁾, s⁽⁰⁾ = \tilde{x}⁽⁰⁾, t⁽⁰⁾ = 1
  while not converge do
    ${t}^{( k )} = \frac{1}{2} ( 1 + \sqrt{1 + 4 {( {t}^{( k - 1 )} )}^{2}} )$
    Der(s^{(k-1)} = ∇ₛ_{_{(k−1)}} [symbol]  (s^{(k-1)}, y) + γ(s^{(k-1)} − G_{θ}(s^{(k-1)}))
    \tilde{x}^{(k)} ← s^{(k-1)} − η · Der(s^{(k-1)})
    ${s}^{( k )} ← {\tilde{x}}^{( k )} + \frac{{t}^{( k - 1 ) - 1}}{{t}^{( k )}} ( {\tilde{x}}^{( k )} - {\tilde{x}}^{( k - 1 )} )$
    if ||\tilde{x}^{(k)} − \tilde{x}^{(k-1)}|| ≤ tol · ||\tilde{x}^{(k-1)}|| then
      break
    end if
  end while
```

10. The image enhancing method of claim 8, further comprising providing a report indicative of at least one classification of the second image.

11. A computerized image processing method, comprising:
applying, by a processor, a machine learning framework to translate a first image to a second image, wherein the first image comprises a source domain and the second image comprises a target domain; and
saving, by the processor, the second image to a memory.

12. The computerized image processing method of claim 11, wherein the machine learning framework is a generative adversarial network.

13. The computerized image processing method of claim 12, wherein the machine learning framework is an optimal transport-guided unpaired generative adversarial network.

14. The computerized image processing method of claim 13, further comprising utilizing the second image to assist in diagnosis of retinopathy.

15. The computerized image processing method of claim 14, further comprising classifying, by the processor, the second image into at least one of a plurality of classifications, the plurality of classifications includes a normal classification and one or more disorder classifications, wherein the one or more disorder classifications includes at least one of: age-related macular degeneration (AMD), Diabetic Retinopathy (DR), glaucoma, or Retinal Vein Occlusion (RVO).

16. The computerized image processing method of claim 15, wherein the machine learning framework utilizes the equation:
$${max}_{{G}_{θ}} {min}_{{D}_{w}} {\sum }_{i = 1}^{n} [ {αℒ}_{d} ( {y}_{i} , {G}_{θ} ( {y}_{i} ) ) + {βℒ}_{idt} ( {x}_{i} , {G}_{θ} ( {x}_{i} ) ) ] + {𝒲}_{1} ( {ℙ}_{X} , {ℙ}_{{G}_{θ} ( Y )} ) ,$$
where G_{θ} is a generator parameterized by θ;
D_{w}, a discriminator, is a 1-Lipschitz function parameterized by w;
L_{d} and L_{idt} denotes a domain transport cost and an identity constraint cost, respectively; and
α, β are weight parameters of a domain loss and an identity loss, respectively.

17. The computerized image processing method of claim 16, wherein the translating comprises use of an algorithm of the form:
Algorithm 1 OT-Guided Unpaired Image-to-Image Translation.
```
Require: The learning rate η, the batch size m, the gradient penalty weight
  λ, the consistency loss weight α ≤ 1, the identity loss weight β.
Require: Initial discriminator parameters w₀, initial generator parameters θ₀.
  while not converge do
    $Sample a batch of low - quality images y = {\{ {y}_{i} \}}_{i = 1}^{m} ∼ {ℙ}_{Y} with {\{ {g}_{i} \}}_{i = 1}^{m} .$
    $Sample a batch of high - quality images x = {\{ {x}_{i} \}}_{i = 1}^{m} ∼ {ℙ}_{X} with {\{ {g}_{i} \}}_{i = 1}^{m} .$
    for i = 1, . . . , m do
      Sample a random ϵ~U[0, 1].
      \tilde{x}ᵢ ← G_{θ}(yᵢ)
      \hat{x}ᵢ ← ϵxᵢ + (1 − ϵ) \tilde{x}ᵢ
      ${ℒ}_{{D}_{w}} ( i ) ← {D}_{w} ( {\tilde{x}}_{i} ) - {D}_{w} ( {x}_{i} ) + {λ ( {‖ {∇}_{{\hat{x}}_{i}} {D}_{w} ( {\hat{x}}_{i} ) ‖}_{2} - 1 )}_{+}^{2}$
    end for
    $w ← w + η \cdot RMSProp ( w , {∇}_{w} \frac{1}{m} {\sum }_{i = 1}^{m} {ℒ}_{{D}_{w}} ( i ) )$
    ${ℒ}_{{G}_{θ}} ← \frac{1}{m} {\sum }_{i = 1}^{m} - {D}_{w} ( {G}_{θ} ( y ) ) + {αℒ}_{d} ( y , {G}_{θ} ( y ) ) + {βℒ}_{idt} ( x , {G}_{θ} ( x ) )$
    θ ← θ − η · RMSProp(θ, ∇_{θ} [symbol] G_{θ})
  end while
```

18. The computerized image processing method of claim 17, wherein the machine learning framework utilizes the equation:
$$\hat{x} = {arg min}_{x} {𝔼}_{x} [ ℒ ( x , y ) ] + γ R ( x ) with R ( x ) = \frac{1}{2} {x}^{T} ( x - {G}_{θ} ( x ) ) ,$$
where γ controls a regularization strength, and L denotes a multi-scale structural similarity loss.

19. The computerized image processing method of claim 16, wherein the translating comprises use of an algorithm of the form:
Algorithm 2 Regularization by Enhancing.
```
Require: The step size η, regularization strength γ, tolerance tol, Generator
  G_{θ}
Require: Initial \tilde{x}⁽⁰⁾, s⁽⁰⁾ = \tilde{x}⁽⁰⁾, t⁽⁰⁾ = 1
  while not converge do
    ${t}^{( k )} = \frac{1}{2} ( 1 + \sqrt{1 + 4 {( {t}^{( k - 1 )} )}^{2}} )$
    Der(s^{(k-1)} = ∇ₛ_{_{(k−1)}} [symbol]  (s^{(k-1)}, y) + γ(s^{(k-1)} − G_{θ}(s^{(k-1)}))
    \tilde{x}^{(k)} ← s^{(k-1)} − η · Der(s^{(k-1)})
    ${s}^{( k )} ← {\tilde{x}}^{( k )} + \frac{{t}^{( k - 1 ) - 1}}{{t}^{( k )}} ( {\tilde{x}}^{( k )} - {\tilde{x}}^{( k - 1 )} )$
    if ||\tilde{x}^{(k)} − \tilde{x}^{(k-1)}|| ≤ tol · ||\tilde{x}^{(k-1)}|| then
      break
    end if
  end while
```

20. The computerized image processing method of claim 19, wherein the first image is a retinal fundus photography image.
